(19) United States
(12) Patent Application Publication
(10) Pub. No.: US 2007/0250117 A1
(43) Pub. Date: Oct. 25, 2007

(54) Medical diagnostic tool

(76) Inventor: Tsong Yun Kwong, Wembley Park (GB)

Correspondence Address: J. WILEY HORTON, ESQUIRE;Pennington, Moore, Wilkinson, Bell & Dunbar, P.A., Post Office Box 10092, Tallahassee, FL, 32302-2095, US

(21) Appl. No.: 11/804,260

(22) Filed: May 17, 2007

(30) Foreign Application Priority Data
Nov. 17, 2005 (GB) PCT/GB05/04447

(51) Int. Cl. A61B 17/08

(52) U.S. Cl. 606/219

(57) ABSTRACT

A diagnostics tool for use in clinical diagnosis, particularly but not exclusively, neurological diagnosis. The diagnostic tool includes a tendon reflex hammer comprising a percussive head adjacent an end of a shaft. The tool also includes an acoustic tone or vibration generating device capable of producing a predetermined frequency or frequencies.

The present invention relates to a diagnostics tool for use in clinical diagnosis, particularly but not exclusively, neurological diagnosis.

During neurological examination and diagnosis, it is necessary for the clinicians to use a selection of tools to carry out a range of tests on the patient. Research has shown that a comprehensive set of tools can weigh in excess of 5 kg, whilst it is also extremely expensive to purchase. As a result, it is a common practice for clinicians to carry only a basic kit consisting of a tendon hammer, an opthalmoscope, a tuning fork, some neurotips and a pen torch. Even this basic kit is not inexpensive and weighs around one kg. The use of such a basic kit has the disadvantage that the clinician, when on a hospital round or home visit for example, relies on borrowing equipment as he goes or making do. This leads to an inefficient use of time and can lead to inaccurate diagnosis. Very often from the experience of the applicant, there is often a general lack of these tools in hospital wards, frequently forcing clinicians to spend valuable time hunting these tools down in wards, which may even be on another hospital wing

The present invention seeks to provide a solution to these disadvantages by providing a diagnostics tool which incorporates more than one of the aforesaid diagnostic devices.

According to the present invention there is provided medical diagnostics tool including a tendon reflex hammer comprising a percussive head adjacent an end of a shaft, the tool including a generating device adapted to generate at least one acoustic tone and/or vibration having a predetermined frequency.

Preferably, the generating device is adapted to generate selectively a plurality of acoustic tones, each having a discrete predetermined frequency, and may also be adapted to generate a vibration of a predetermined frequency. Furthermore, the generating device may also be adapted to generate a plurality of vibrations each having a discrete predetermined frequency. The frequency or frequencies may be one or more of 128 hertz, 256 hertz, 512 hertz and 1024 hertz.

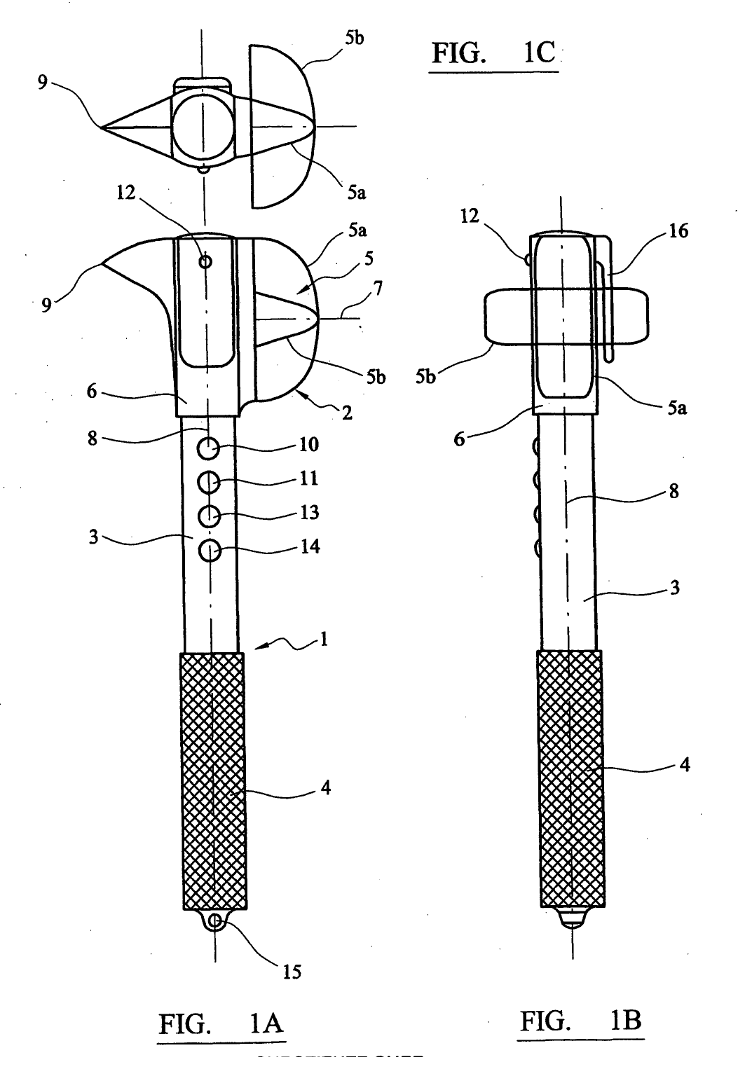

FIG. 1A shows a front view of a diagnostics tool

FIG. 1B shows a side view of the tool, and

FIG. 1C shows an end view of the tool.

Referring now to the drawing, there is shown a diagnostics tool for neurological examination and diagnosis which comprises a handle 1 having on one end a percussion head 2. The handle 1 comprises a substantially hollow tubular shaft formed of two shaft sections 3, 4 telescopically movable one relative to the other. The shaft section 4 has a knurled finish to provide a hand grip. As shown in the drawing, the two shaft sections 3, 4 are in the extended position. Although not shown, locking means is provided to lock the shaft sections in the extended position. The tubular shaft sections 3, 4 are formed of aluminium or stainless steel in this embodiment, or other materials such as a plastics materials may be utilised.

The percussion head 2 may be formed of a plastics material bonded to the end of the shaft section 3, but may also be formed of a metal such as aluminium or stainless-steel. The head includes an arcuate striking surface or edge 5 which is pivotable relative to the main body 6 of the head about an axis 7 perpendicular to the axis 8 of the handle between a position, shown in outline as reference 5a, in which the plane of the striking edge 5 lies parallel to the axis 8 of the handle, or to a position, shown in outline as reference Sb, displaced by 90° in which the plane of the arcuate edge 5 lies at right angles to the axis 8 of the handle.

On its side remote from the striking edge 5, the percussion head 1 includes a pointed end which forms a reflex scraper 9.

Located in the interior of the handle there is one or more piezo-electric devices powered by an electric battery (not shown) located in the handle adjacent the percussion head. In this embodiment, the piezo electric device is designed to emit an acoustic tone and/or vibration at either 128, 256, 512 or 1024 hertz. Each frequency has its own operating button, or on/off switch 10, 11, switch 10 initiating the generation of the 128 hertz frequency and switch 11 initiating the generation of the 256 hertz frequency.

1. A medical diagnostics tool including a tendon reflex hammer comprising a percussive head adjacent an end of a shaft, a generating device adapted to generate at least one acoustic tone and/or vibration having a predetermined frequency and an opthalmoscope.

2. A tool according to claim 1 wherein the generating device is adapted to generate selectively a plurality of acoustic tones or vibrations, each having a discrete predetermined frequency or sequence thereof.

3. A tool according to claim 1, including an indicator light adapted to indicate that a frequency has been generated.

4. A tool according to claim 3, wherein the colour of the indicator light is variable, the colour being representative of the frequency being generated.

5. A tool according to claim 1, including a function to allow the frequency to decay at a set predetermined rate.

6. A tool according to claim 4, further including red and white lights generated selectively by a light-emitting diode.

7. A tool according to claim 1, wherein the shaft is telescopic, having a plurality of relatively movable shaft sections.

8. A tool according to claim 7, including means to lock the shaft sections securely together in a selected extended position.

9. A tool according to claim 1, wherein the shaft includes a connection by which at least one further implement is detachably connectable to the shaft.

10. A tool according to claim 1, further including one or more devices selected from the group of, a further light, a peak flow meter, a stethoscope, a blood pressure monitor, saturations monitor, a thermometer and an arm carrying a point is pivotally mounted on said handle so as to be movable to a position spaced from a second point on the tool to enable a two point discrimination test to be carried out.

11. A tool according to claim 1, wherein the percussive head has a planar arcuate striking edge, the striking edge being pivotal relative to the shaft so that the plane of the striking edge is selectively adjustable to lie parallel to the axis of the shaft or normal thereto.

12. A medical diagnostics tool including a tendon reflex hammer comprising a percussive head adjacent to an end of a shaft, the tool including a generating device adapted to generate at least one acoustic tone or vibration having a predetermined frequency, and wherein said percussive head has a planar arcuate striking edge, said striking edge being pivotal relative to said shaft so that the plane of said striking edge is selectively adjustable to lie parallel to the axis of said shaft or normal thereto.

13. A tool according to claim 12, wherein the generating device is adapted to generate selectively a plurality of acoustic tones or vibrations, each having a discrete predetermined frequency or sequence thereof.

14. A tool according to claim 12, including an indicator light adapted to indicate that a frequency has been generated.

15. A tool according to claim 14, wherein the color of the indicator light is variable, the color being representative of the frequency being generated.

16. A tool according to claim 12, including a function to allow the frequency to decay at a set predetermined rate.

17. A tool according to claim 15, further including red and white lights generated selectively by a light-emitting diode.

18. A tool according to claim 12, wherein the shaft is telescopic, having a plurality of relatively movable shaft sections.

19. A tool according to claim 18, including means to lock the shaft sections securely together in a selected extended portion.

20. A tool according to claim 12, wherein the shaft includes a connection by which at least one further implement is detachably connectable to the shaft.
